(19) United States
(12) Patent Application Publication
(10) Pub. No.: US 2012/0035541 A1
(43) Pub. Date: Feb. 9, 2012

(54) SINGLE USE SAFETY SYRINGE

(76) Inventor: Graeme Walton, New South Wales (AU)

(21) Appl. No.: 13/143,862

(22) PCT Filed: Jan. 8, 2010
(86) PCT No.: PCT/AU2010/000014

§ 371 Date: Sep. 23, 2011

(30) Foreign Application Priority Data
Jan. 9, 2009 (AU) 2009 900 074
Jan. 19, 2009 (AU) 2009 900 196

(51) Int. Cl. A61M 5/50

(52) U.S. Cl. 604/110

(57) ABSTRACT

A single use safety syringe (10) has a barrel (12) having a leading end (14) and a trailing end (16). A needle 18 is sealably, but removably, attached to an opening in the leading end. A plunger (20) is slidably received through an opening in the trailing end. Upon completion of an injection, the plunger (20) engages the needle (18) and the needle can then be manually re-tracted into the barrel (12). A locking tab assembly (42, 44, 50, 52) is securely connected to a main body of the barrel (12) and forms the opening in the trailing end (16). The locking tab assembly includes two locking fingers (78, 80). The plunger (20) has a locking plate (41) which includes two locking ports (86, 88). The locking fingers (78, 80) and locking ports (86, 88) form a needle locking assembly. The locking fingers (78, 80) are adapted to engage by a hooked fit the locking ports (86, 88) when the needle (18) is fully retracted after use to prevent any subsequent movement of the plunger (20) with the engaged needle towards the leading end. Upon such engagement, a holding plate (36) secured to the plunger (20) below the locking plate (41) butts up against the bottom of holding rings (82, 84) of the locking tab assembly to prevent any subsequent movement of the plunger with the engaged needle towards the trailing end.

### TECHNICAL FIELD

The present invention relates to a single use, safety syringe. In particular, the present invention relates to a syringe having a manually retractable needle that can be retained safely, after a single use, within the barrel of the syringe by a needle locking assembly.

### BACKGROUND ART

Contaminated syringes pose a very serious danger to the health of persons who handle them, and many devices and techniques have been proposed to protect such persons from direct contact with, or injury by, contaminated syringes. Preventing needle stick injury to persons using syringes to collect, transfer and/or inject liquids for medical and other uses is especially important where there is a risk of transmitting blood borne diseases, such as Hepatitis B and Human Immunodeficiency Virus (HIV). Many of the proposed solutions provide an arrangement for retracting the contaminated needle into the barrel of the syringe so that both can be disposed of at one time without the person coming into contact with the needle. Such retractable needle syringes can be so arranged as to retract their needles either automatically or manually. With automatically retractable syringes, when an injection has been administered, the plunger is further depressed and deactivates a locking mechanism which normally locks the hub of the needle within the front or leading end of the barrel under tension of a spring or other such biasing means. Deactivation of the locking mechanism allows the needle to retract into the barrel under the influence of the spring. Similarly, with manually retractable syringes, when an injection has been administered, the plunger becomes locked to the hub of the needle by a mechanism that allows the needle to retract into the barrel as the plunger is withdrawn or pulled away from the front of the barrel.

However, with both automatically and manually retractable needle syringes, measures need to be taken to ensure that the plunger is not able to be, by deliberate or accidental means, completely withdrawn from the rear or trailing end of the barrel or, if possible, reloaded into the barrel to cause the needle to re-emerge from the front of the barrel, and thereby expose a person to the risk of contact with contaminated liquid or needle stick injury.

One approach to ensuring that the plunger cannot be reused in these ways after the needle has been manually retracted into the barrel is to snap off the emerging end of the plunger at a pre-weakened point of the plunger adjacent the opening to the rear of the barrel, but this is not entirely satisfactory as access may still be gained to the snap off point of the plunger retained within the barrel and there is no or little resistance to moving the so retained plunger relative to the barrel once such access has been obtained.

### DISCLOSURE OF INVENTION

It is an object of the present invention to provide a single use, safety syringe that is arranged to retract its needle manually, and which has a needle locking assembly that can retain a retracted needle within the barrel of the syringe in so reliable a way as to prevent any subsequent movement of the plunger to which the needle is attached relative to the barrel, and to restrict access to the snap off point of the plunger.

According to the present invention, there is provided a single use safety syringe comprising a barrel having a leading end and a trailing end, a needle sealably, but removably, attached to an opening in the leading end, a plunger slidably received through an opening in the trailing end, wherein, upon completion of an injection, the plunger engages the needle and the needle can then be manually retracted into the barrel, a locking tab assembly securely connected to a main body of the barrel and forming the opening in the trailing end, the locking tab assembly including at least one first locking member and at least one first holding member when assuming a locking-ready position, wherein the or each first locking member is adapted to engage by either of a hooked fit or a snapped fit a respective one or more second locking member connected with the plunger when the needle is fully retracted to prevent any subsequent movement of the plunger with the engaged needle towards the leading end, and wherein, upon such engagement, the or each first holding member is adapted to butt up against a respective one or more second holding member connected with the plunger to prevent any subsequent movement of the plunger with the engaged needle towards the trailing end.

In a first preferred embodiment, when engagement is by a hooked fit, the or each first locking member is a locking finger, and the respective one or more second locking member is a locking port.

Alternatively, the or each first locking member is a locking port, and the respective one or more second locking member is a locking finger.

It is preferred that there are two locking fingers and there are two respective locking ports. Preferably, the or each first holding member is a holding ring, and the respective one or more second holding member is a holding plate.

In one preferred form of this embodiment, the holding plate is adapted to hold a sealing element that slidably seals against the inside surface of the barrel, the holding plate and sealing element forming a piston head of the plunger.

In another preferred form of this embodiment, the two locking ports are formed in a locking plate located between the holding plate and a weakness in a stem of the plunger.

The weakness preferably serves as a snap off point of the plunger when the needle is fully retracted, the weakness being so located along the stem of the plunger that access to the snap off point, after the emerging end of the plunger has been snapped off, is restricted by the locking tab assembly.

In a particularly preferred form of this embodiment, the locking tab assembly has a pair of opposed finger tabs integrally formed with the main body of the barrel, and a pair of top tabs, each pivotally connected to a respective finger tab, wherein, when the top tabs are pivotally opened from the finger tabs, the opening in the trailing end of the barrel is of a first size to allow the plunger to be inserted through that opening for assembly of the syringe prior to its use, and wherein, when the top tabs are pivotally closed against the finger tabs, the opening in the trailing end of the barrel is of a second size less than the first size to allow only a stem of an already inserted plunger to slide through that reduced opening for use of the syringe.

In this embodiment, the top tabs may be pivotally closed against the finger tabs by engagement of an inner guide finger extending from each top tab with a respective guide aperture formed through each finger tab, and by engagement of an outer closing finger extending from each top tab with a respective slot formed at the periphery of opposed collar portions which join together the finger tabs, thereby allowing the locking tab assembly to assume the locking-ready position.

In a second preferred embodiment, when engagement is by a snapped fit, the or each first locking member is a one-way locking tongue, and the respective one or more second locking member is a locking plate.

Alternatively, the respective one or more second locking member is a locking wedge.

It is preferred that there are four locking tongues and there is one locking plate having a circumferential rim that is an inclined plane.

Preferably, the or each first holding member is a holding ring, and the respective one or more second holding member is a holding plate.

In one preferred form of this embodiment, the holding plate is adapted to hold a sealing element that slidably seals against the inside surface of the barrel, the holding plate and sealing element forming a piston head of the plunger.

In another preferred form of this embodiment, the locking plate is located between the holding plate and a weakness in a stem of the plunger.

Alternatively, there are four locking wedges, each one of which is located between the holding plate and the weakness in the stem of the plunger.

The weakness preferably serves as a snap off point of the plunger when the needle is fully retracted, the weakness being so located along the stem of the plunger that access to the snap off point, after the emerging end of the plunger has been snapped off, is restricted by the locking tab assembly.

In a particularly preferred form of this embodiment, the locking tab assembly has a pair of opposed finger tabs integrally formed with the main body of the barrel, and a pair of top tabs, each pivotally connected to a respective finger tab, wherein, when the top tabs are pivotally opened from the finger tabs, the opening in the trailing end of the barrel is of a first size to allow the plunger to be inserted through that opening for assembly of the syringe prior to its use, and wherein, when the top tabs are pivotally closed against the finger tabs, the opening in the trailing end of the barrel is of a second size less than the first size to allow only a stem of an already inserted plunger to slide through that reduced opening for use of the syringe.

In this embodiment, the top tabs may be pivotally closed against the finger tabs by engagement of an inner guide finger extending from each top tab with a respective guide aperture formed through each finger tab, and by engagement of an outer closing finger extending from each top tab with a respective slot formed at the periphery of opposed collar portions which join together the finger tabs, thereby allowing the locking tab assembly to assume the locking-ready position.

### BRIEF DESCRIPTION OF THE DRAWINGS

In order that the invention may be more readily understood and put into practical effect, reference will now be made to the accompanying drawings, in which:

FIG. 1 is an upright front view of a syringe according to one form of a first preferred embodiment of the invention, with the plunger located within the barrel in preparation for the drawing of liquid into the barrel,

FIG. 2 is an upright front view of the barrel with attached needle of the syringe of FIG. 1, with a locking tab assembly of the syringe shown in an artificially separated state,

FIG. 3 is an upright front view of the plunger of the syringe of FIG. 1,

FIG. 4 is a plan view of the syringe of FIG. 1,

FIG. 5 is an upright side view of the barrel with attached needle, and the artificially separated locking tab assembly, of the syringe (less the plunger) shown in FIG. 2,

FIG. 6 is a plan view of the opened locking tab assembly of the syringe of FIG. 1, showing detail of the locking plate and the stem of the plunger,

FIG. 7 is an upright front view of the syringe of FIG. 1 in a final stage of assembly prior to its use, with the plunger being inserted into the barrel, and the top tabs about to be pivotally closed against the finger tabs to allow the locking tab assembly to assume a locking-ready position,

FIG. 8 is an upright side view of the syringe of FIG. 1 after the needle has been fully retracted into the barrel and the part of the plunger that emerges from the barrel has been snapped off,

FIG. 9 is an upright front view of a syringe according to another form of a first preferred embodiment of the invention, with the plunger located within the barrel in preparation for the drawing of liquid into the barrel,

FIG. 10 is a plan view of the opened locking tab assembly of the syringe of FIG. 9, showing detail of the holding plate and the stem of the plunger,

FIG. 11 is an isolated upright front view of the opposed top tabs of the locking tab assembly of the syringe of FIG. 9,

FIG. 12 is an isolated upright side view of one of the top tabs shown in FIG. 11,

FIG. 13 is an upright front view of the syringe of FIG. 9 in a final stage of assembly prior to its use, with the plunger being inserted into the barrel, and the top tabs about to be pivotally closed against the finger tabs to allow the locking tab assembly to assume a locking-ready position,

FIG. 14 is an upright front view of the syringe shown in FIG. 13 after its final stage of assembly and ready for use,

FIG. 15 is an upright front view of the syringe shown in FIG. 14 after a first stage of use in drawing liquid into the barrel of the syringe,

FIG. 16 is an upright front view of the syringe shown in FIG. 15 after a second stage of use in releasing liquid through the needle and allowing the plunger to engage the needle in preparation for the manual retraction of the needle into the barrel,

FIG. 17 is an isolated, partly cut away, perspective view of a lower part of the plunger at a first stage of movement towards the locking tab assembly in a locking-ready position during manual retraction of the needle of the syringe shown in FIG. 16,

FIG. 18 is an isolated, partly cut away, perspective view of the lower part of the plunger shown in FIG. 17 at a second stage of movement during manual retraction of the needle, where the locking fingers from the holding plate are in the process of engaging the locking ports in the locking tab assembly,

FIG. 19 is an isolated, partly cut away, perspective view of the lower part of the plunger shown in FIG. 18 after the second stage of movement, where the locking fingers have engaged the locking ports to activate the needle locking assembly and thereby fully retract the needle within the barrel,

FIG. 20 is an upright front view of the syringe shown in FIG. 16 after a third stage of use in fully retracting the needle into the barrel,

FIG. 21 is an upright side view of the syringe shown in FIG. 20 after a fourth stage of use in snapping off the part of the plunger that emerges from the barrel,

FIG. 22 is an upright front view of a syringe according to one form of a second preferred embodiment of the invention, with the plunger located within the barrel in preparation for the drawing of liquid into the barrel,

FIG. 23 is an upright front view of the barrel with attached needle of the syringe of FIG. 22, with a locking tab assembly of the syringe shown in an artificially separated state,

FIG. 24 is an upright front view of the plunger of the syringe of FIG. 22,

FIG. 25 is a plan view of the syringe of FIG. 22,

FIG. 26 is an upright side view of the barrel with attached needle, and the artificially separated locking tab assembly, of the syringe (less the plunger) shown in FIG. 23,

FIG. 27 is a plan view of the opened locking tab assembly of the syringe of FIG. 22, showing detail of the locking plate and the stem of the plunger,

FIG. 28 is an upright front view of the syringe of FIG. 22 in a final stage of assembly prior to its use, with the plunger being inserted into the barrel, and the top tabs about to be pivotally closed against the finger tabs to allow the locking tab assembly to assume a locking-ready position,

FIG. 29 is an upright side view of the syringe of FIG. 22 after the needle has been fully retracted into the barrel and the part of the plunger that emerges from the barrel has been snapped off,

FIG. 30 is an enlarged view of part of the syringe shown in FIG. 29,

FIG. 31 is an upright front view of the syringe of FIG. 22 in a final stage of assembly prior to its use, with the plunger being inserted into the barrel, and the top tabs about to be pivotally closed against the finger tabs to allow the locking tab assembly to assume a locking-ready position,

FIG. 32 is an upright front view of the syringe shown in FIG. 31 after its final stage of assembly and ready for use,

FIG. 33 is an upright front view of the syringe shown in FIG. 32 after a first stage of use in drawing liquid into the barrel of the syringe,

FIG. 34 is an upright front view of the syringe shown in FIG. 33 after a second stage of use in releasing liquid through the needle and allowing the plunger to engage the needle in preparation for the manual retraction of the needle into the barrel,

FIG. 35 is an isolated, partly cut away, perspective view of a lower part of the plunger at a first stage of movement towards the locking tab assembly in a locking-ready position during manual retraction of the needle of the syringe shown in FIG. 34,

FIG. 36 is an isolated, partly cut away, perspective view of the lower part of the plunger shown in FIG. 35 at a second stage of movement during manual retraction of the needle, where the locking tongues are in the process of engaging the locking plate,

FIG. 37 is an isolated, partly cut away, perspective view of the lower part of the plunger shown in FIG. 36 after the second stage of movement, where the locking tongues have engaged the locking plate to activate the needle locking assembly and thereby fully retract the needle within the barrel,

FIG. 38 is an upright front view of the syringe shown in FIG. 34 after a third stage of use in fully retracting the needle into the barrel, and

FIG. 39 is an upright side view of the syringe shown in FIG. 38 after a fourth stage of use in snapping off the part of the plunger that emerges from the barrel.

### MODES FOR CARRYING OUT THE INVENTION

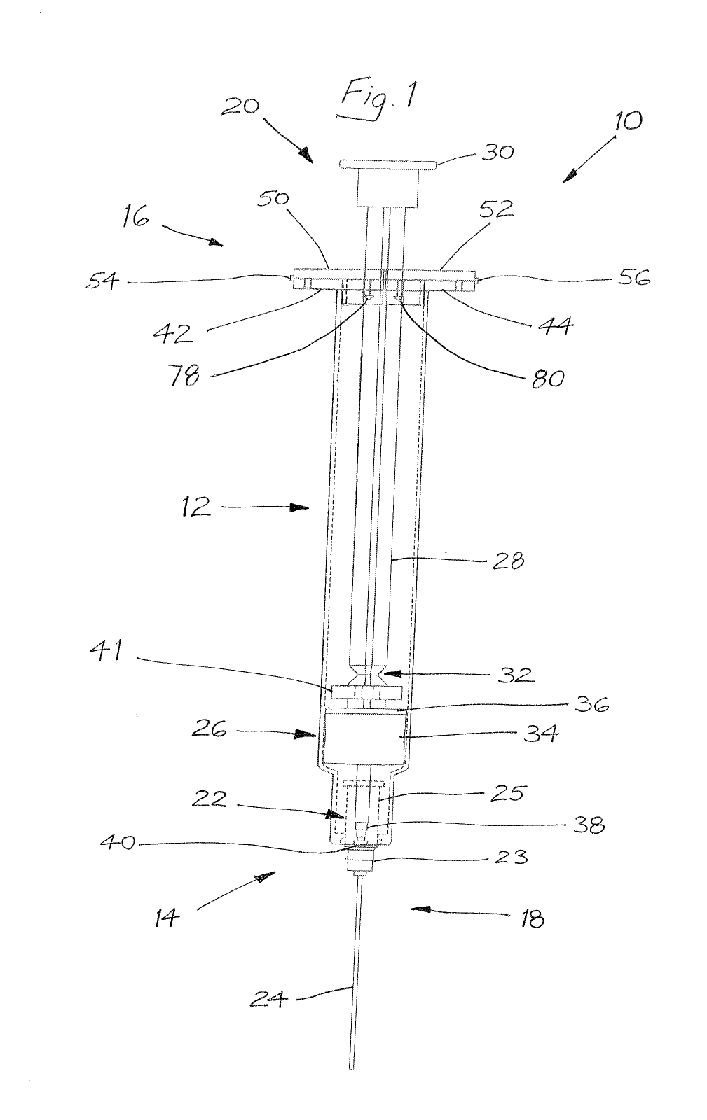

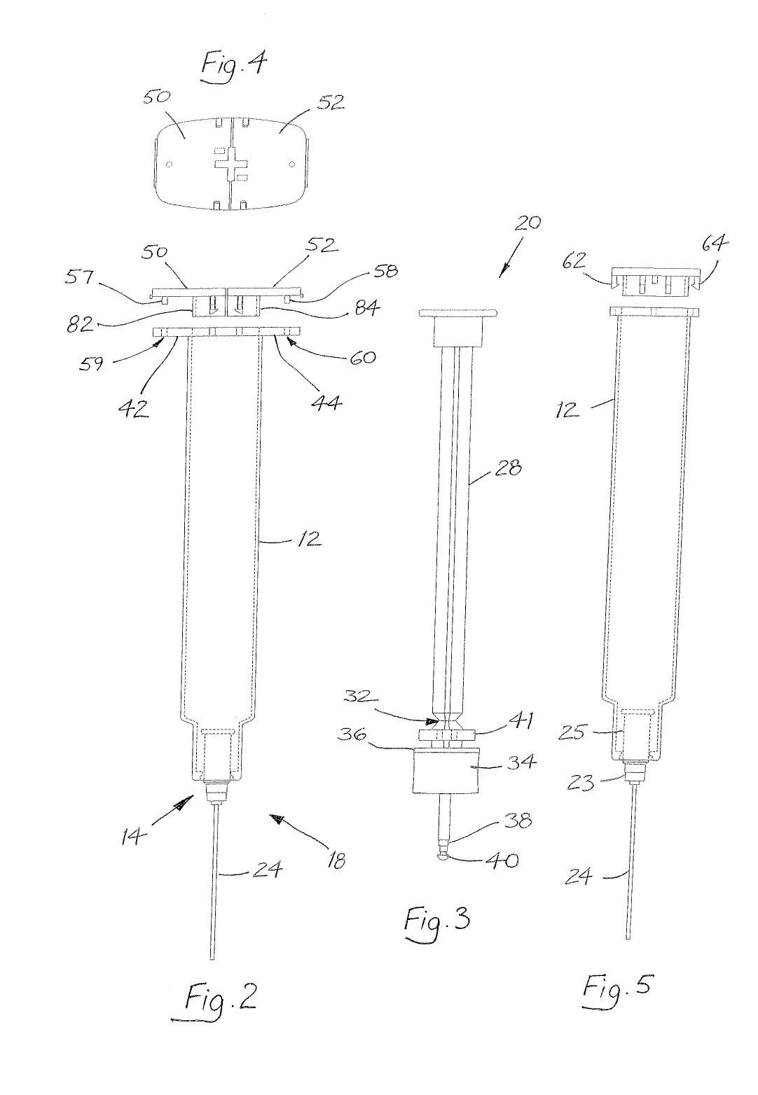

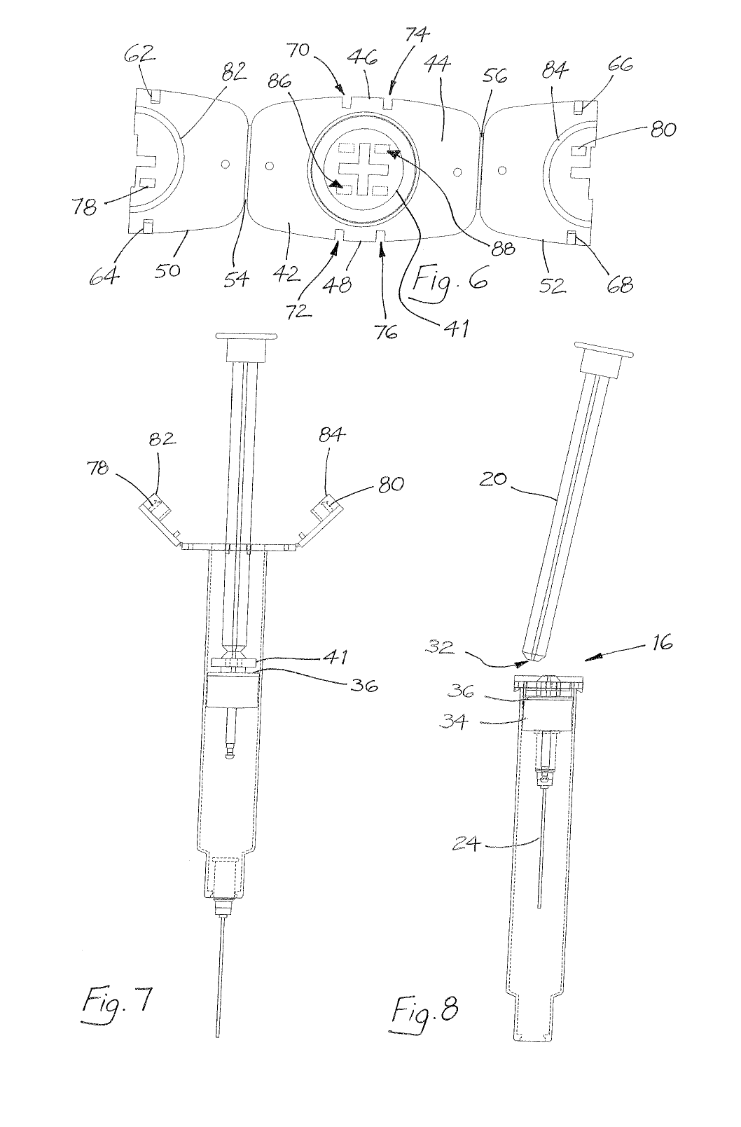

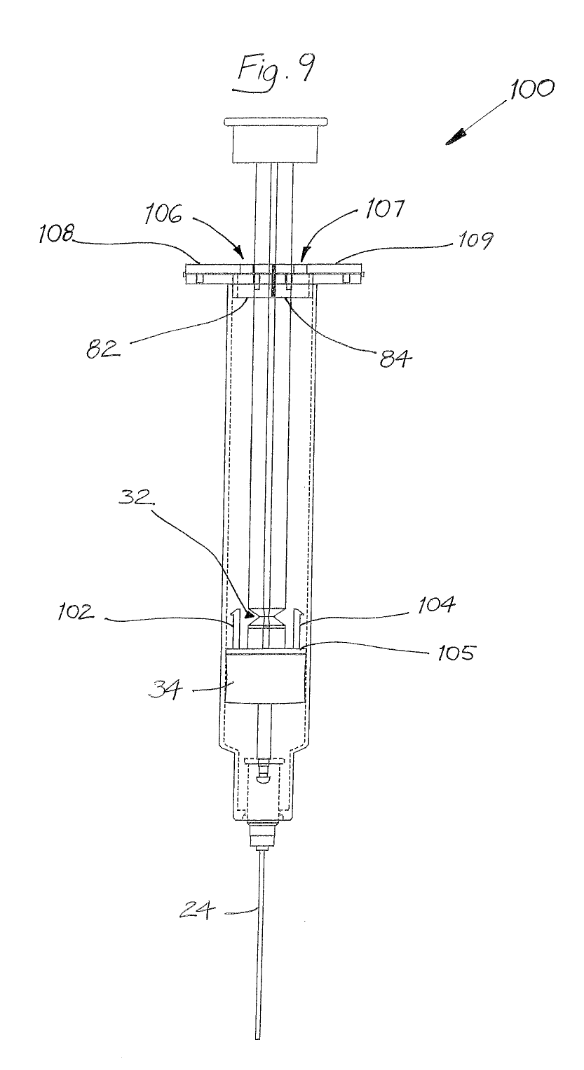

The syringe 10 shown in FIGS. 1 to 8 is one form of a first preferred embodiment of the invention, and has a hollow barrel 12 made of a clear plastic material with a leading end 14 and a trailing end 16, a needle 18 sealably, but removably, attached to an opening in the leading end 14, and a plunger 20 slidably received through an opening in the trailing end 16.

The needle 18 has a needle hub 22 and a stainless steel needle tip 24 mounted to the hub 22. The hub 22 has an external part 23 located outside the barrel 12 and an internal part 25 located inside the barrel 12, the internal part 25 having a hollow bore that provides for liquid communication between the barrel 12 and a passageway through the external part 23 of the hub leading to the needle tip 24. There is a flexible annular shoulder (not shown) having a constricting inner diameter within the passageway through the external part of the needle hub 22.

The plunger 20 has a piston head 26, a stem 28, and a finger pad 30. The stem 28 is formed of four radially extending, equally spaced apart, webs, except where there is a localised narrowing or weakness 32 of the stem 28 located just above the piston head 26. The weakness 32, when required, serves as a snap off point of the plunger 20.

The piston head 26 has a rubber gasket or liquid tight sealing element 34 that slidably seals against the inside surface of the barrel 12, and a holding plate 36 for the sealing element 34, and there is a stepped shaft 38 extending forwardly of the sealing element 34.

The shaft 38 has a domed catch member 40 at its free end. The catch member 40 has an outer diameter that, when required, allows it to engage by a forced snap fit behind the annular shoulder within the passageway through the external part 23 of the needle hub 22.

There is also a locking plate 41 securely located on the stem 28 between the weakness 32 and the holding plate 36 of the piston head 26. The structure and function of the locking plate 41 will be described in detail later in the specification.

The opening in the trailing end 16 of the barrel 12 through which the plunger 20 is slidably received is formed by a locking tab assembly that is closed into a locking-ready position prior to use of the syringe 10. The locking tab assembly has a pair of integrally formed finger tabs 42, 44, each extending outwardly in opposed directions from the main body of the barrel 12, but also joined together by collar portions 46, 48. The locking tab assembly also has a pair of pivotable top tabs 50, 52, each connected by an integral hinge 54, 56 to an outermost edge of a respective finger tab 42, 44. In an alternative embodiment, the top tabs may be separate from the finger tabs and may be clipped into position. When the top tabs 50, 52 are pivotally opened from the finger tabs 42, 44, the opening in the trailing end 16 of the barrel 12 is at its optimum size and of a circular shape to allow the piston head 26 of the plunger 20 to be inserted therethrough for assembly of the syringe prior to its use. When the top tabs 50, 52 are pivotally closed against the finger tabs 42, 44, the opening in the trailing end 16 of the barrel 12 is reduced in size and of a cruciform shape to allow only the webbed stem 28 of the already inserted plunger 20 to slide therethrough. A semicircular spacer or holding ring 82, 84 extending downwardly from each top tab 50, 52 engages by interference fit a respective semicircular inner side wall portion of the opening in the trailing end 16 of the barrel 12. For closure of the top tabs 50, 52 against the finger tabs 42, 44 to occur, and for the locking tab assembly to assume a locking-ready position, an inner guide finger 57, 58 extending from each top tab 50, 52 engages by interference fit a respective guide aperture 59, 60 formed through each finger tab 42, 44, and a pair of outer closing fingers 62, 64 and 66, 68 extending from each top tab 50, 52 engages by hooked fit a respective slot 70, 72 and 74, 76 formed at the periphery of the collar portions 46, 48 of the finger tabs 42, 44.

In the locking-ready position, the locking tab assembly forms part of a needle locking assembly for reliably retaining a fully manually retracted needle, after use of the syringe, say, for an injection, within the barrel of the syringe. The needle locking assembly includes a locking finger 78, 80 extending downwardly from each top tab 50, 52 and which terminates in a barb located a distance from each top tab 50, 52 that is just above the distance each holding ring 82, 84 extends downwardly from each top tab 50, 52. The locking fingers 78, 80 are located within a respective semicircular holding ring 82, 84.

The needle locking assembly also includes locking ports 86, 88 formed in the locking plate 41. A locking finger 78, 80 engages by hooked fit a respective locking port 86, 88 of the locking plate 41 when the needle is fully manually retracted after use. When so fully retracted, the peripheral top surface of the holding plate 36 butts up against the bottom surface of each holding ring 82, 84 because the outer diameter of the locking plate 41 is just less than the inner diameter of each holding ring 82, 84.

The part of the plunger 20 that emerges from the opening in the trailing end 16 may then be snapped off. Because of the location of the weakness or snap off point 32 along the stem 28 of the plunger 20, access to the snap off point, after the emerging end of the plunger has been snapped off, is restricted by the locking tab assembly.

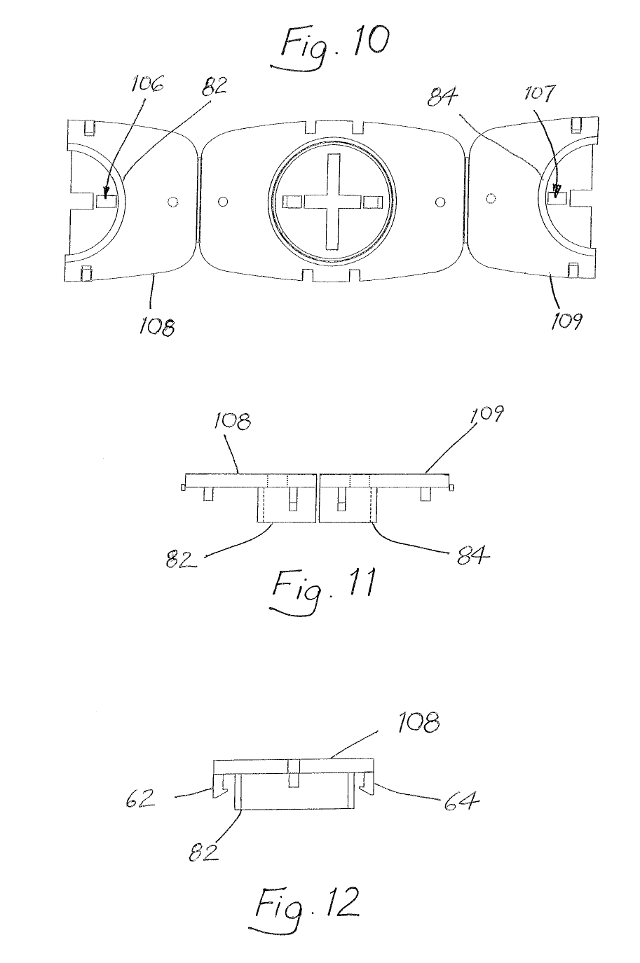

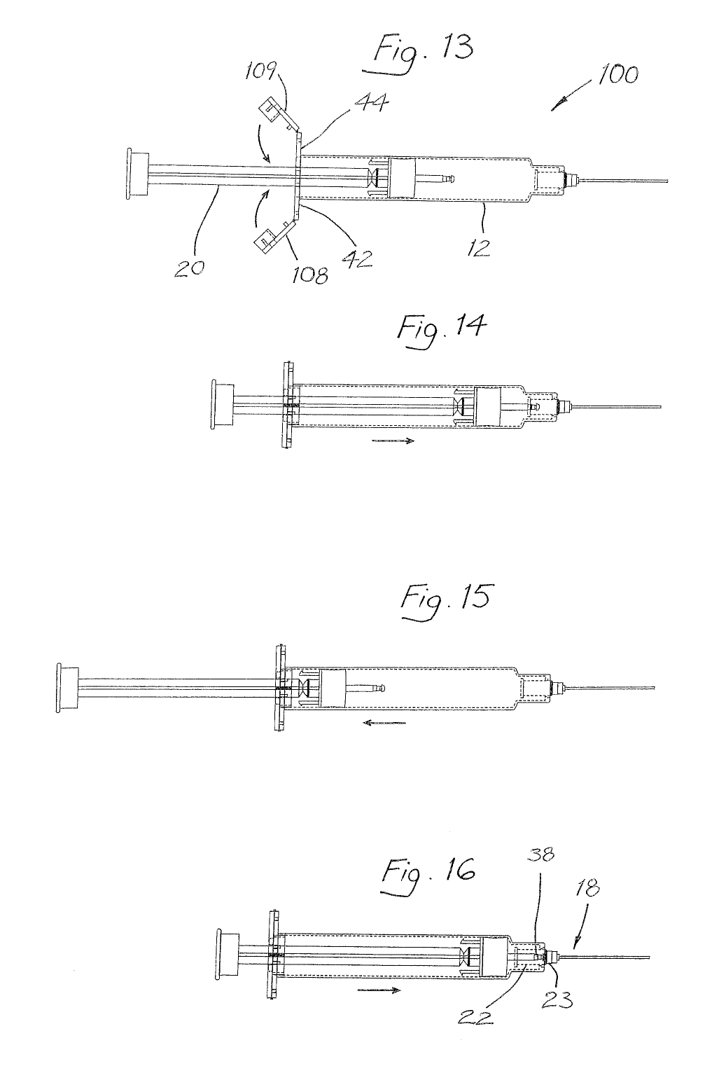

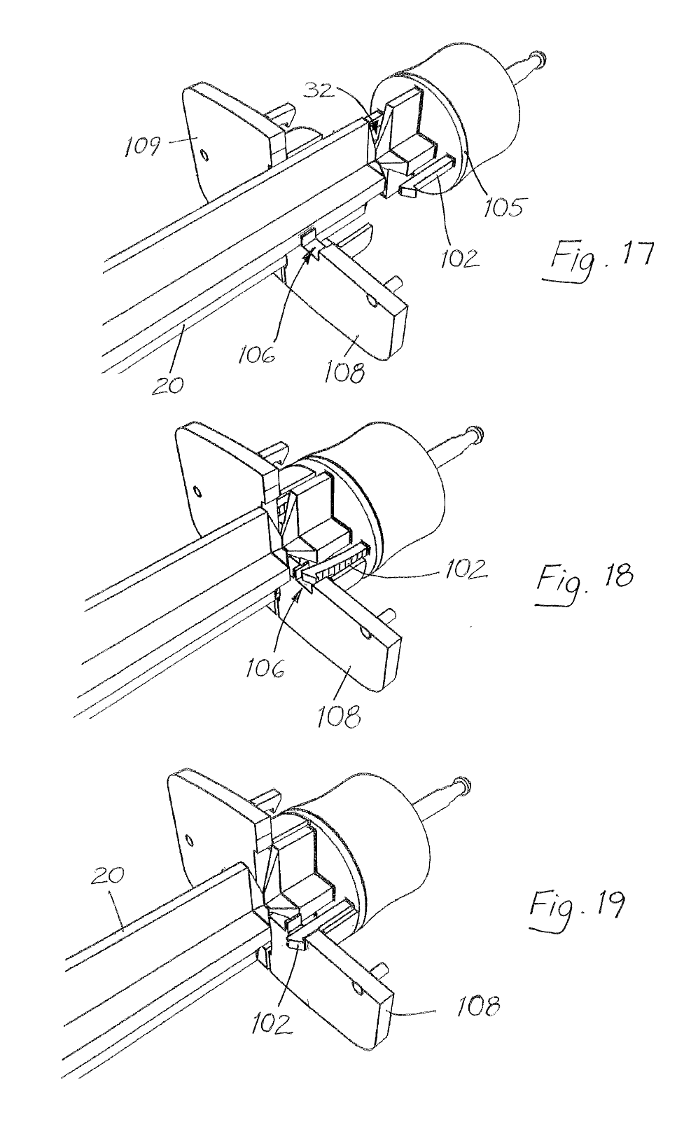

The syringe 100 shown in FIGS. 9 to 21 is another form of a first preferred embodiment of the invention, and is similar in structure and function to the syringe 10 shown in FIGS. 1 to 8, and so like features have been accorded like numerals in the Figures. However, syringe 100 does not have a locking plate 41 with locking ports 86, 88, and does not have locking fingers 78, 80 extending downwardly from the top tabs 50, 52. Rather, syringe 100 has locking fingers 102, 104 extending upwardly from its holding plate 105, each of which terminate in a barb located a distance from the holding plate 105 that is just above the distance its stem weakness 32 is from the holding plate 105. The syringe 100 also has locking ports 106, 107 formed in each top tab 108, 109. The locking ports 106, 107 are located within a respective semicircular holding ring 82, 84. A locking finger 102, 104 engages by hooked fit a respective locking port 106, 107 of the top tabs 108, 109 when the needle is fully manually retracted after use. When so fully retracted, the peripheral top surface of the holding plate 105 butts up against the bottom surface of each holding ring 82, 84.

For both syringes 10 and 100, the plunger 20, to which the retracted needle 18 is attached, is locked firmly in the fully retracted position by the engagement of the locking fingers with the locking ports (to prevent any subsequent downward movement of the plunger relative to the barrel), and by the holding plate 36 (for syringe 10) or the holding plate 105 (for syringe 100) butting up against the holding rings of the locking tab assembly in its locking-ready position (to prevent any subsequent upward movement of the plunger relative to the barrel). The secure means by which the locking tab assembly is closed into its locking-ready position prior to use of the syringe 10, 100 provides the necessary strength to prevent such downward and upward movement when the needle is fully manually retracted after use.

In the stepped sequence of events shown in FIGS. 13 to 21, FIG. 13 shows a final stage of assembly of the syringe 100 prior to its use, with the plunger 20 inserted into the barrel 12, and the top tabs 108, 109 about to be pivotally closed against the finger tabs 42, 44 to allow the locking tab assembly to assume a locking-ready position.

FIG. 14 shows the syringe 100 with the plunger 20 pushed into the barrel 12 in preparation for the drawing of liquid into the barrel of the syringe.

FIG. 15 shows the syringe 100 after the plunger 20 has been pulled back to draw liquid into the barrel 12 of the syringe.

FIG. 16 shows the syringe 100 after the plunger 20 has been pushed back into the barrel 12 to release liquid through the needle 18 for the purpose of injection and the stepped shaft 38 has been engaged by a forced snap fit to the external part 23 of the needle hub 22.

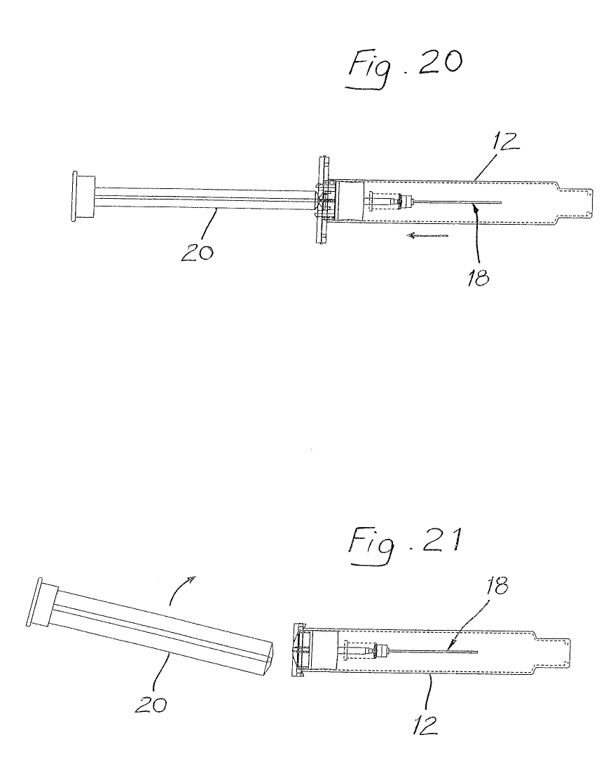

FIG. 20 shows the syringe 100 after the plunger 20, to which the needle 18 is attached, has been pulled back to fully retract the needle into the barrel 12 and reliably retain the so retracted needle within the barrel by activation of the needle locking assembly.

FIG. 21 shows the syringe 100 after the emerging part of the plunger 20 has been snapped off, whereupon both the snapped off plunger and the barrel 12 containing the retracted needle 18 may be disposed of safely.

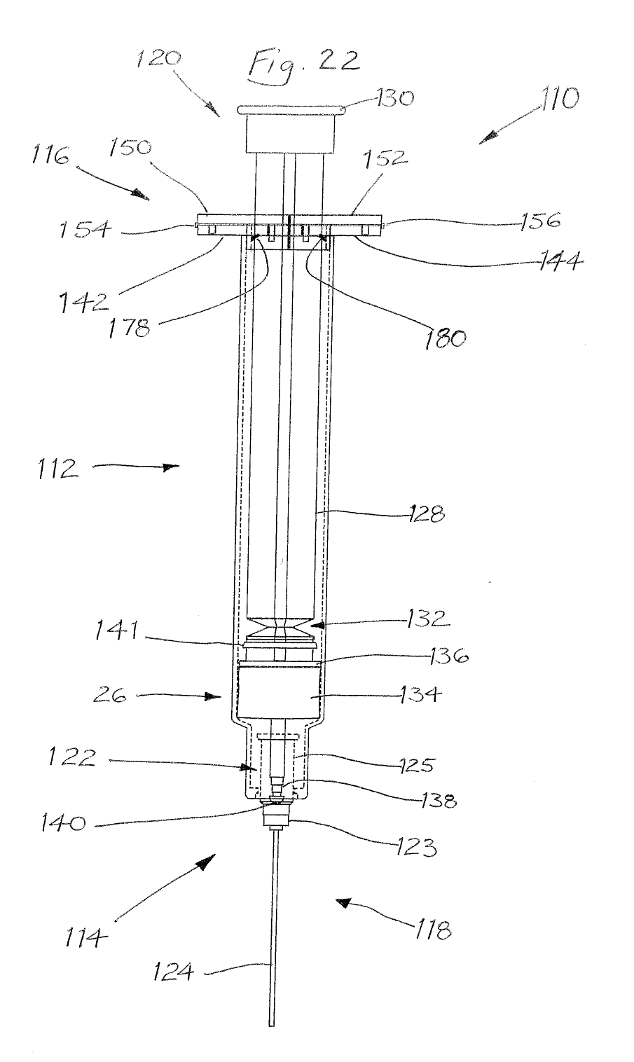

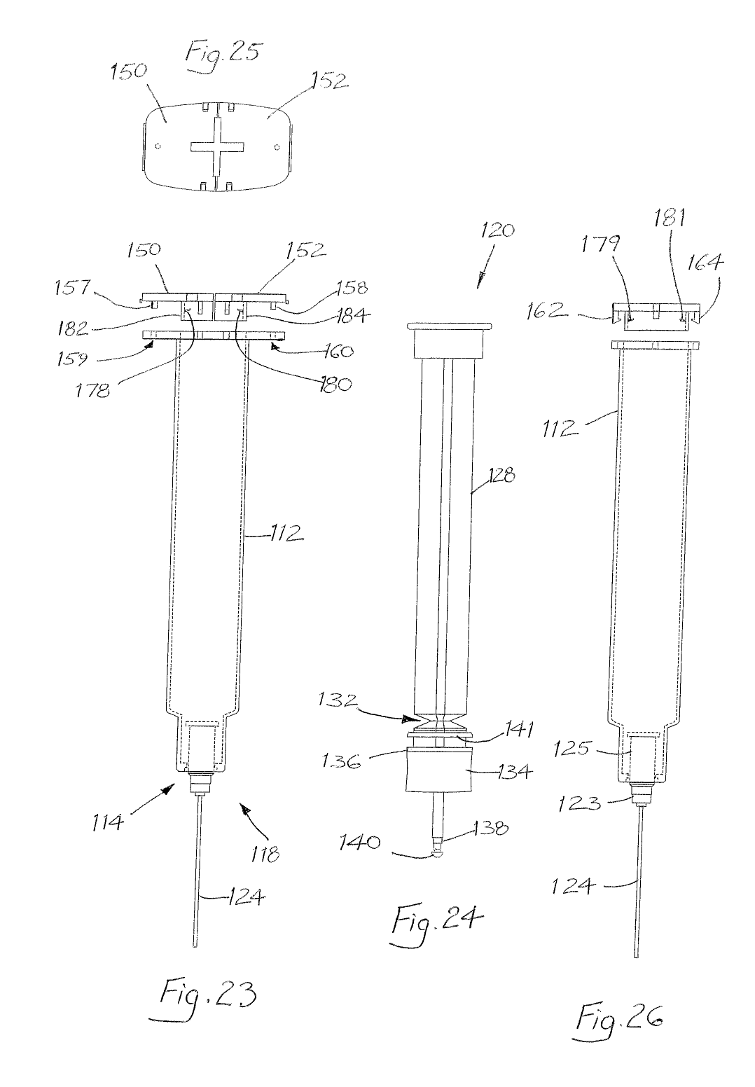

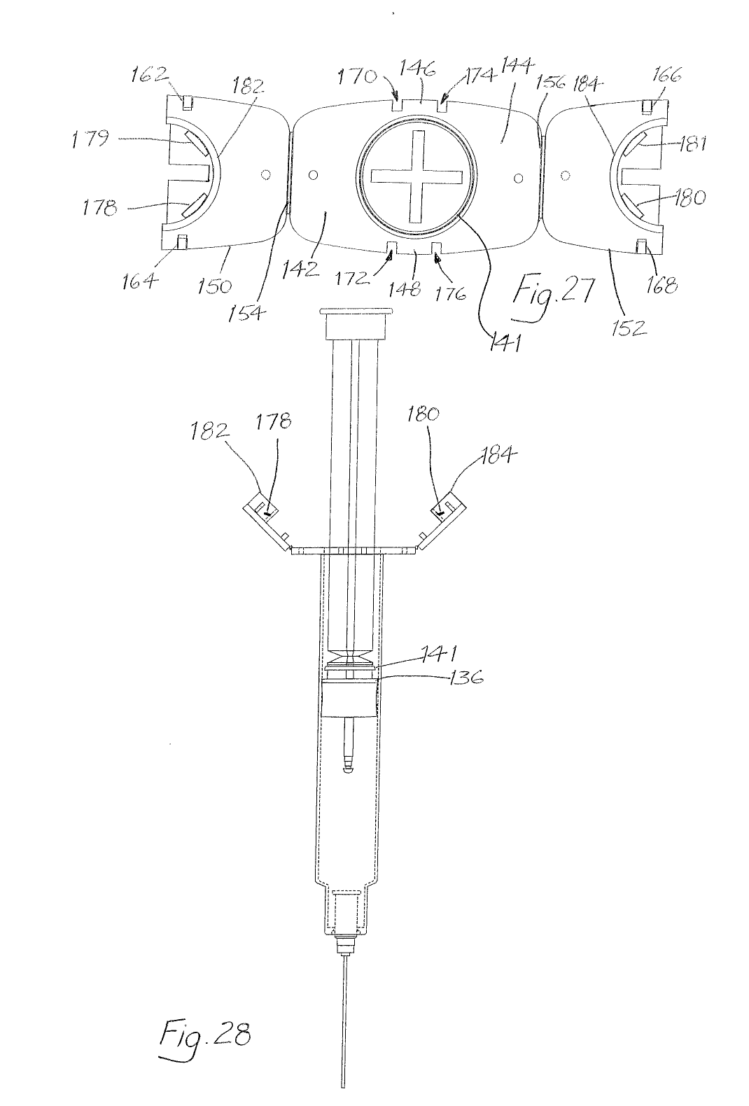

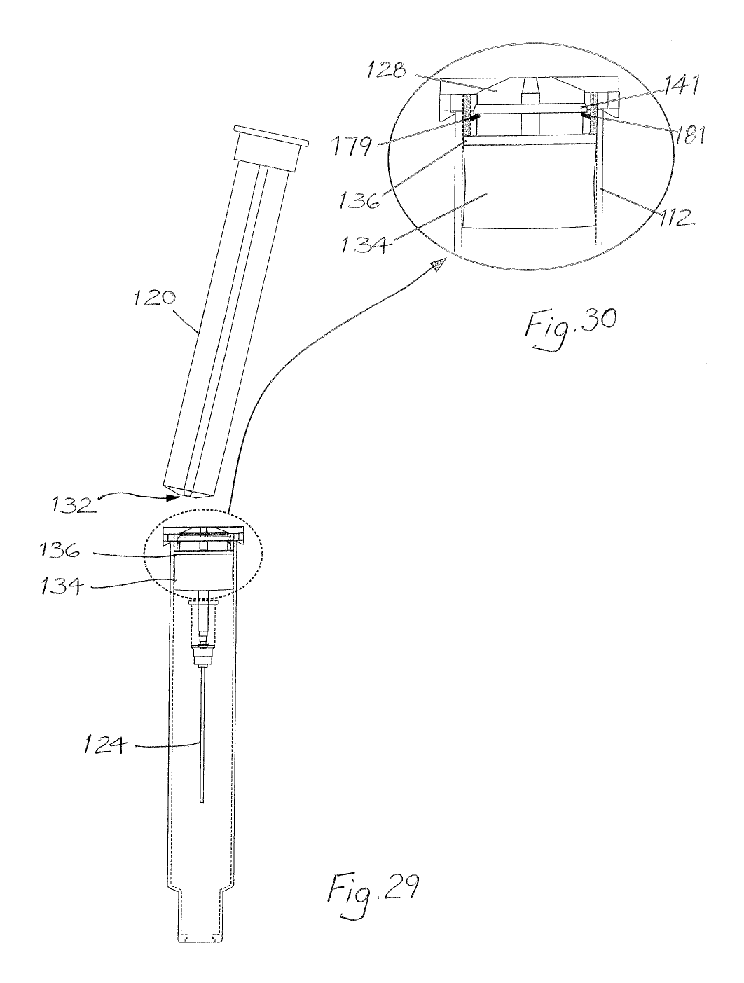

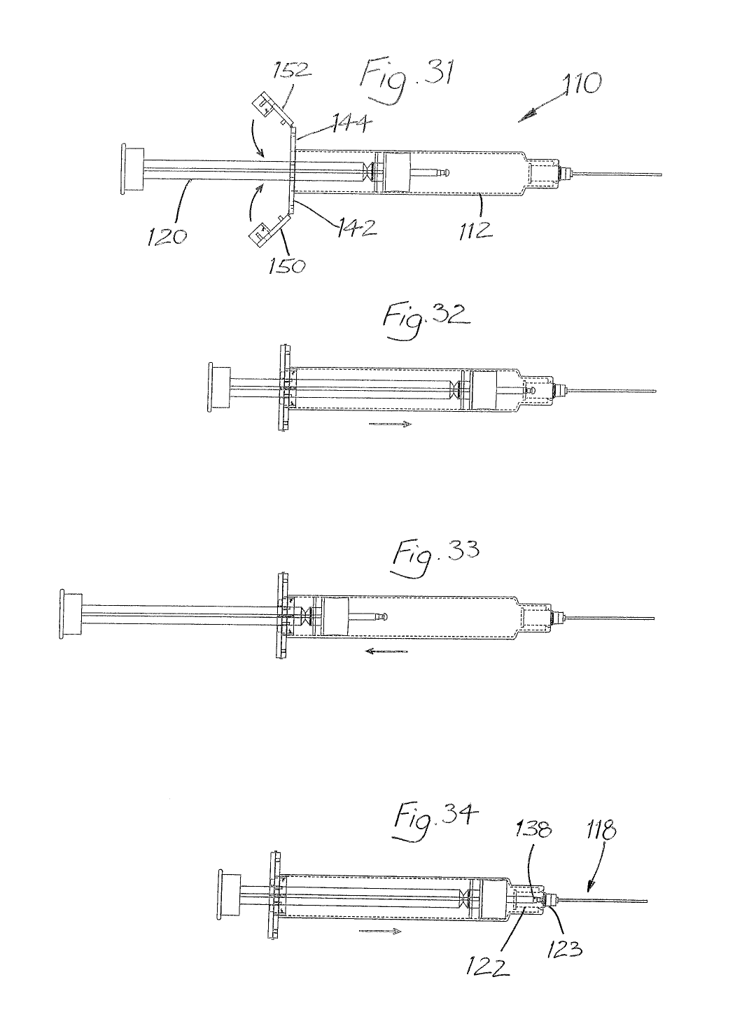

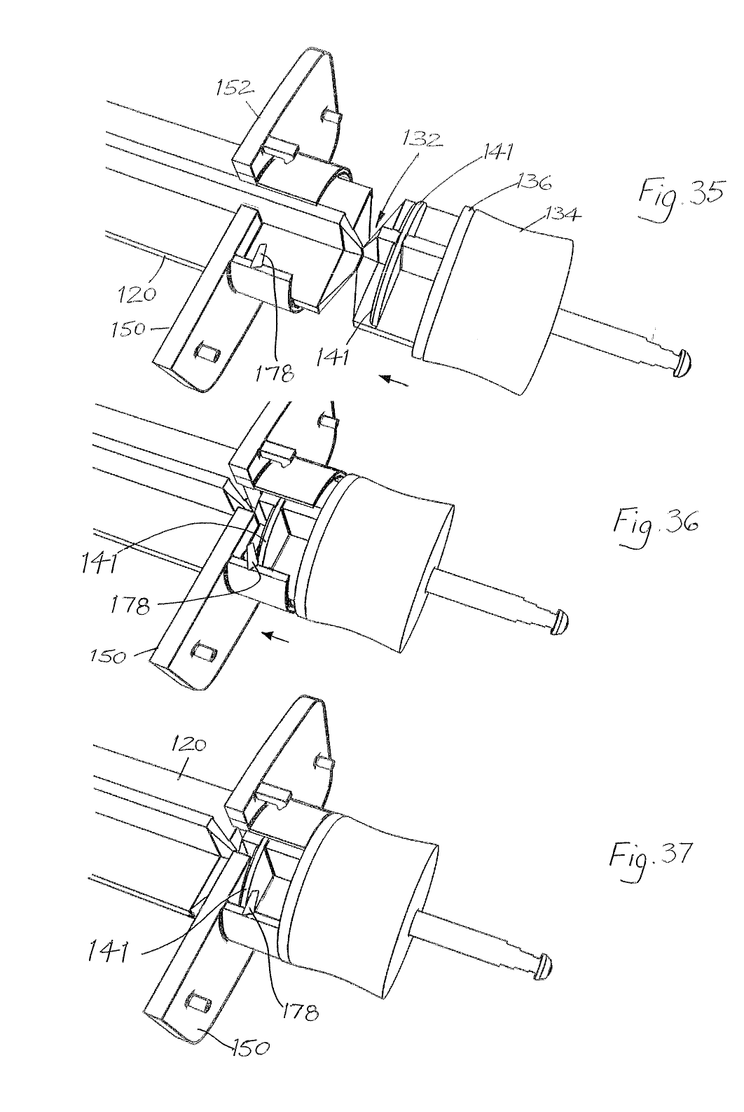

The syringe 110 shown in FIGS. 22 to 39 is one form of a second preferred embodiment of the invention, and has a hollow barrel 112 made of a clear plastic material with a leading end 114 and a trailing end 116, a needle 118 sealably, but removably, attached to an opening in the leading end 114, and a plunger 120 slidably received through an opening in the trailing end 116.

The needle 118 has a needle hub 122 and a stainless steel needle tip 124 mounted to the hub 122. The hub 122 has an external part 123 located outside the barrel 112 and an internal part 125 located inside the barrel 112, the internal part 125 having a hollow bore that provides for liquid communication between the barrel 112 and a passageway through the external part 123 of the hub leading to the needle tip 124. There is a flexible annular shoulder (not shown) having a constricting inner diameter within the passageway through the external part of the needle hub 122.

The plunger 120 has a piston head 126, a stem 128, and a finger pad 130. The stem 128 is formed of four radially extending, equally spaced apart, webs, except where there is a localised narrowing or weakness 132 of the stem 128 located just above the piston head 126. The weakness 132, when required, serves as a snap off point of the plunger 120.

The piston head 126 has a rubber gasket or liquid tight sealing element 134 that slidably seals against the inside surface of the barrel 112, and a holding plate 136 for the sealing element 134, and there is a stepped shaft 138 extending forwardly of the sealing element 134.

The shaft 138 has a domed catch member 140 at its free end. The catch member 140 has an outer diameter that, when required, allows it to engage by a forced snap fit behind the annular shoulder within the passageway through the external part 123 of the needle hub 122.

There is also a locking plate 141 securely located on the stem 128 between the weakness 132 and the holding plate 136 of the piston head 126. The structure and function of the locking plate 141 will be described in detail later in the specification.

The opening in the trailing end 116 of the barrel 112 through which the plunger 120 is slidably received is formed by a locking tab assembly that is closed into a locking-ready position prior to use of the syringe 110. The locking tab assembly has a pair of integrally formed finger tabs 142, 144, each extending outwardly in opposed directions from the main body of the barrel 112, but also joined together by collar portions 146, 148. The locking tab assembly also has a pair of pivotable top tabs 150, 152, each connected by an integral hinge 154, 156 to an outermost edge of a respective finger tab 142, 144. In an alternative embodiment, the top tabs may be separate from the finger tabs and may be clipped into position. When the top tabs 150, 152 are pivotally opened from the finger tabs 142, 144, the opening in the trailing end 116 of the barrel 112 is at its optimum size and of a circular shape to allow the piston head 126 of the plunger 120 to be inserted therethrough for assembly of the syringe prior to its use. When the top tabs 150, 152 are pivotally closed against the finger tabs 142, 144, the opening in the trailing end 116 of the barrel 112 is reduced in size and of a cruciform shape to allow only the webbed stem 128 of the already inserted plunger 120 to slide therethrough. A semicircular spacer or holding ring 182, 184 extending downwardly from each top tab 150, 152 engages by interference fit a respective semicircular inner side wall portion of the opening in the trailing end 116 of the barrel 112. For closure of the top tabs 150, 152 against the finger tabs 142, 144 to occur, and for the locking tab assembly to assume a locking-ready position, an inner guide finger 157, 158 extending from each top tab 150, 152 engages by interference fit a respective guide aperture 159, 160 formed through each finger tab 142, 144, and a pair of outer closing fingers 162, 164 and 166, 168 extending from each top tab 150, 152 engages by hooked fit a respective slot 170, 172 and 174, 176 formed at the periphery of the collar portions 146, 148 of the finger tabs 142, 144.

In the locking-ready position, the locking tab assembly forms part of a needle locking assembly for reliably retaining a fully manually retracted needle, after use of the syringe, say, for an injection, within the barrel of the syringe. The needle locking assembly includes a pair of spaced apart, one way, locking tongues 178, 179 and 180, 181 extending from a starting position inwardly and upwardly from the inner side wall of a respective pair of semicircular holding rings 182, 184 of each top tab 150, 152 and which are structurally biased to flex upwardly but not downwardly.

The needle locking assembly also includes a locking plate 144 having a circumferential rim that is an inclined plane. A locking tongue 178, 179, 180, 181 engages by snapped fit behind a respective trailing end portion of the locking plate 141 when the needle is fully manually retracted after use. When so fully retracted, the peripheral top surface of the holding plate 136 butts up against the bottom surface of each holding ring 182, 184 because the outer diameter of the locking plate 141 is just less than the inner diameter of each holding ring 182, 184.

In another form (not shown) of this embodiment, the locking plate 141 may be replaced by four locking wedges, each formed between adjacent webs of the stem 128, and located between the weakness 132 and the holding plate 136. Each locking wedge has a sharp end nearest the weakness 132 and a blunt end nearest the holding plate 136 so as to define an upwardly inclined plane against which a respective locking tongue 178, 179, 180, 181 flexes progressively more upwardly as the plunger approaches the end of its range of retraction. When each locking tongue simultaneously passes the blunt end of its respective locking wedge, it is snapped back to its inward and upward starting position.

The locking tongues engage the inclined plane at the rim of the locking plate 141 in a similar manner (see especially FIGS. 35 to 37).

The part of the plunger 120 that emerges from the opening in the trailing end 116 may then be snapped off. Because of the location of the weakness or snap off point 132 along the stem 128 of the plunger 120, access to the snap off point, after the emerging end of the plunger has been snapped off, is restricted by the locking tab assembly.

For syringe 110, the plunger 120, to which the retracted needle 118 is attached, is locked firmly in the fully retracted position by the engagement of the locking tongues with the locking plate (or with the locking wedges for the aforementioned, but not shown, other form of this embodiment) to prevent any subsequent downward movement of the plunger relative to the barrel, and by the holding plate 136 butting up against the holding rings of the locking tab assembly in its locking-ready position to prevent any subsequent upward movement of the plunger relative to the barrel. The secure means by which the locking tab assembly is closed into its locking-ready position prior to use of the syringe 110 provides the necessary strength to prevent such downward and upward movement when the needle is fully manually retracted after use.

In the stepped sequence of events shown in FIGS. 31 to 39, FIG. 31 shows a final stage of assembly of the syringe 110 prior to its use, with the plunger 120 inserted into the barrel 112, and the top tabs 150, 152 about to be pivotally closed against the finger tabs 142, 144 to allow the locking tab assembly to assume a locking-ready position.

FIG. 32 shows the syringe 110 with the plunger 120 pushed into the barrel 112 in preparation for the drawing of liquid into the barrel of the syringe.

FIG. 33 shows the syringe 110 after the plunger 120 has been pulled back to draw liquid into the barrel 112 of the syringe.

FIG. 34 shows the syringe 110 after the plunger 120 has been pushed back into the barrel 112 to release liquid through the needle 118 for the purpose of injection and the stepped shaft 138 has been engaged by a forced snap fit to the external part 123 of the needle hub 122.

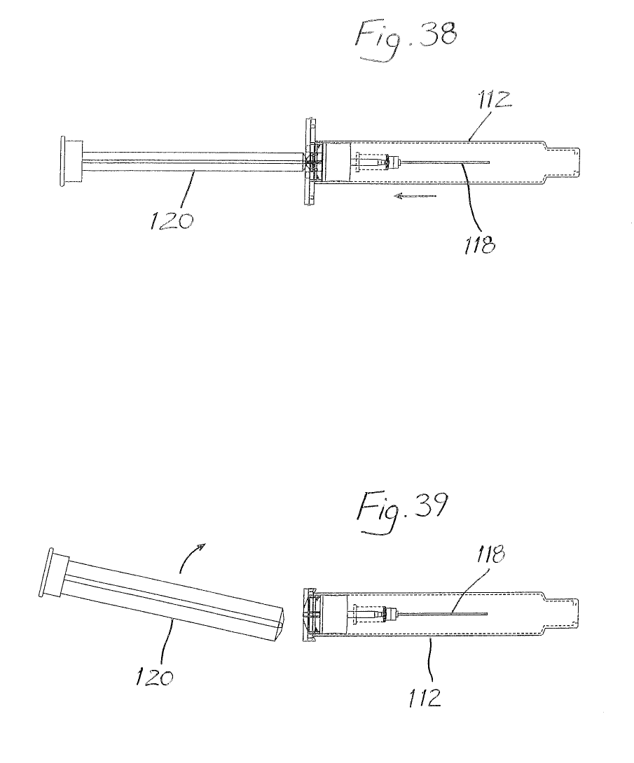

FIG. 38 shows the syringe 110 after the plunger 120, to which the needle 118 is attached, has been pulled back to fully retract the needle into the barrel 112 and reliably retain the so retracted needle within the barrel by activation of the needle locking assembly.

FIG. 39 shows the syringe 110 after the emerging part of the plunger 120 has been snapped off, whereupon both the snapped off plunger and the barrel 112 containing the retracted needle 118 may be disposed of safely.

It will be readily apparent to persons skilled in the art that various modifications may be made in details of design, construction and use of the single use safety syringe described herein without departing from the scope or ambit of the present invention.

### INDUSTRIAL APPLICABILITY

The single use safety syringe as described in various forms and embodiments in the specification is particularly useful for preventing needle stick injury to persons using syringes to collect, transfer and/or inject liquids for medical or other uses, and is especially applicable to reducing the risk of transmitting blood borne diseases.

1. A single use safety syringe comprising a barrel having a leading end and a trailing end, a needle sealably, but removably, attached to an opening in the leading end, a plunger slidably received through an opening in the trailing end, wherein, upon completion of an injection, the plunger engages the needle and the needle can then be manually retracted into the barrel, a locking tab assembly securely connected to a main body of the barrel and forming the opening in the trailing end, the locking tab assembly including at least one first locking member and at least one first holding member when assuming a locking-ready position, wherein the or each first locking member is adapted to engage by either of a hooked fit or a snapped fit a respective one or more second locking member connected with the plunger when the needle is fully retracted to prevent any subsequent movement of the plunger with the engaged needle towards the leading end, and wherein, upon such engagement, the or each first holding member is adapted to butt up against a respective one or more second holding member connected with the plunger to prevent any subsequent movement of the plunger with the engaged needle towards the trailing end.

2. The single use safety syringe of claim 1 wherein, when engagement is by a hooked fit, the or each first locking member is a locking finger, and the respective one or more second locking member is a locking port.

3. The single use safety syringe of claim 1 wherein, when engagement is by a hooked fit, the or each first locking member is a locking port, and the respective one or more second locking member is a locking finger.

4. The single use safety syringe of claim 1, wherein when engagement is by a snapped fit, the or each first locking member is a one-way locking tongue, and the respective one or more second locking member is a locking plate.

5. The single use safety syringe of claim 1 wherein, when engagement is by a snapped fit, the respective one or more second locking member is a locking wedge.

6. The single use safety syringe of claim 2 wherein the or each first holding member is a holding ring, and the respective one or more second holding member is a holding plate.

7. The single use safety syringe of claim 6 wherein the holding plate is adapted to hold a sealing element that slidably seals against the inside surface of the barrel, the holding plate and sealing element forming a piston head of the plunger.

8. The single use safety syringe of claim 6 wherein the or each locking port is formed in a locking plate located between the holding plate and a weakness in a stem of the plunger.

9. The single use safety syringe of claim 8 wherein the weakness serves as a snap off point of the plunger when the needle is fully retracted, the weakness being so located along the stem of the plunger that access to the snap off point, after the emerging end of the plunger has been snapped off, is restricted by the locking tab assembly.

10. The single use safety syringe of claim 1 wherein the locking tab assembly has a pair of opposed finger tabs integrally formed with the main body of the barrel, and a pair of top tabs, each pivotally connected to a respective finger tab, wherein, when the top tabs are pivotally opened from the finger tabs, the opening in the trailing end of the barrel is of a first size to allow the plunger to be inserted through that opening for assembly of the syringe prior to its use, and wherein, when the top tabs are pivotally closed against the finger tabs, the opening in the trailing end of the barrel is of a second size less than the first size to allow only a stem of an already inserted plunger to slide through that reduced opening for use of the syringe.

11. The single use safety syringe of claim 4 wherein the or each first holding member is a holding ring, and the respective one or more second holding member is a holding plate.

12. The single use safety syringe of claim 11 wherein the holding plate is adapted to hold a sealing element that slidably seals against the inside surface of the barrel, the holding plate and sealing element forming a piston head of the plunger.

13. The single use safety syringe of claim 11 wherein the locking plate is located between the holding plate and a weakness in a stem of the plunger.

14. The single use safety syringe of claim 5 wherein the or each first holding member is a holding ring, and the respective one or more second holding member is a holding plate.

15. The single use safety syringe of claim 14 wherein there are a plurality of locking wedges, each one of which is located between the holding plate and the weakness in the stem of the plunger.
